(19) United States
(12) Patent Application Publication
(10) Pub. No.: US 2024/0241028 A1
(43) Pub. Date: Jul. 18, 2024

(54) A METHOD OF DETECTING A TARGET MICROPARTICLE

(71) Applicant: SBT Instruments A/S, Herlev (DK)

(72) Inventors: Gustav Skands, Herlev (DK); Sara Paulsen, Herlev (DK); Peter Jordal, Herlev (DK)

(73) Assignee: SBT Instruments A/S, Herlev (DK)

(21) Appl. No.: 18/559,013

(22) PCT Filed: May 4, 2022
(86) PCT No.: PCT/EP2022/061902

§ 371 Date: Nov. 3, 2023

(30) Foreign Application Priority Data
May 4, 2021 (EP) 21171990.1

(51) Int. Cl. G01N 15/1031; G01N 15/10

(52) U.S. Cl. CPC G01N 15/1031; G01N 15/1023; G01N 2015/1006

(57) ABSTRACT

The present invention relates to a method of detecting a target microparticle in a fluid in a microfluidic particle analysis device comprising a measuring channel with a first electrode and a second electrode defining an operating space between and being in electrical connection with an electric current source and a device for monitoring an electrical signal from the first and/or the second electrode. A sample fluid suspected of containing a target microparticle exposing an identification binding partner is mixed with a recognition binding partner component to provide a complex of the recognition binding partner and the identification binding partner, and the complex is labelled with electrically conducting nanoparticles; before optionally adjusting the conductivity to be in the range of 5,000 μS/cm to 50,000 μS/cm and applying a flow of the suspension to the measuring channel of the microfluidic particle analysis device; before applying a current to create an electric field in the operating space and monitoring an electrical signal between the first and the second electrode to detect target microparticles labelled with the electrically conducting nanoparticles. The method is suited to detect a pathogenic bacterium in a water or another fluid.

### FIELD OF THE INVENTION

The present invention relates to a method of detecting a target microparticle in a fluid. In the method, the target microparticle is detected by electrical impedance spectroscopy (EIS) in a microfluidic device. The method is useful in the detection and quantification of specific pathogens as target microparticles.

### BACKGROUND

Impedance spectroscopy is well-described as a label-free method to detect and quantify bacteria and other microparticles in a liquid. For example, the review article by Cheung et al. 2010 (Cytometry Part A, 2010, 77A: 648-666) summarises the background knowledge within the use of impedance flow cytometry in microfluidic systems.

Gawad et al. (Lab Chip, 2004, 4: 241-251) present theoretical considerations for a microfluidic flow cytometer using EIS to analyse cells. Characterisation of cells suspended in KCl solutions of 12,880 μS/cm conductivity is suggested, but no practical examples are shown.

The work of Gawad et al. is implemented into practice by Cheung et al. 2005 (Cytometry Part A, 2005, 65A:124-132). Cheung at al. 2005 study differentiation of red blood cells and derived components and beads of comparable sizes (i.e. about 5 μm in diameter). The fabrication and test of a microfluidic device are demonstrated, and it is shown how EIS using two different frequencies can be performed using the device. The device uses a flow-rate of 10 mm/s, and cells are suspended in phosphate buffered saline of high conductivity.

Houssin et al. (IEEE SENSORS 2009 Conference, 396-399) report the use of EIS in a microdevice for analysing oocysts of a parasite of the species Cryptosporidium parvum in water of low conductivity. David et al. (Biotechnology and Bioengineering, 2011, 109: 483-492) provide a comparison between flow cytometry and microfluidic EIS.

Exemplary microfluidic devices for detecting and quantifying bacteria in drinking water and other liquids are disclosed in WO 2016/116535 and WO 2019/025613.

However, neither of the above prior art disclosures allow detection of specific particles, e.g. pathogenic bacteria, in a population of particles. Clausen et al. (Sensors 2018, 18, 3496; doi:10.3390/s18103496) demonstrated how it is possible to differentiate Escherichia coli from Staphylococcus aureus in low conductivity saline solutions (1×PBS diluted to 1/20 with Milli-Q water). In low conductivity saline solutions, it becomes easier to distinguish bacteria from other particles, but the usefulness of this approach is limited to distinguishing E. coli from S. aureus.

Bertelsen et al. (Sensors 2020, 20, 6339; doi:10.3390/s20216339) demonstrated how it was possible to use impedance flow cytometry to differentiate between inactivated and viable bacteria cells, also in low conductivity saline solutions.

WO 2016/087460 suggests how target particles may be labelled with metallic nanoparticles in order to aid in the detection of the target particles from an electrical signal in a microfluidic system.

As evident from the prior art there is a need for utilising EIS in the detection of specific bacteria, especially within a mixed matrix of bacteria, and it is an object of the invention to provide a method of detecting a specific pathogen using EIS.

### SUMMARY

The present invention relates to a method of detecting a target microparticle in a fluid, the method comprising:
providing a microfluidic particle analysis device comprising a measuring channel having a cross-sectional dimension in the range of 1 μm to 70 μm and a sensor system for detecting a particle, which comprises a first electrode and a second electrode defining an operating space between the first electrode and the second electrode, which first and second electrodes are in electrical connection via an electric circuit comprising an alternating current source and a device for monitoring an electrical signal from the first and/or the second electrode;
providing a sample fluid suspected of containing the target microparticle, which target microparticle exposes an identification binding partner;
providing a recognition binding component comprising a recognition binding partner having a binding affinity for the identification binding partner;
mixing the recognition binding partner component with the sample fluid to provide an application suspension comprising a complex of the recognition binding partner and the identification binding partner;
labelling the complex of the recognition binding partner and the identification binding partner with electrically conducting nanoparticles;
adjusting the conductivity of the sample fluid or the application suspension to be in the range of 5,000 μS/cm to 50,000 μS/cm, if the conductivity of the sample fluid or the application suspension is below 5,000 μS/cm;
applying a flow of the application suspension to the measuring channel of the microfluidic particle analysis device; and
applying an alternating current from the current source to create an electric field in the operating space and monitoring an electrical signal between the first and the second electrode to detect target microparticles labelled with the electrically conducting nanoparticles from a phase of the electrical signal, and optionally also an amplitude of the electrical signal.

In the present method a sample fluid suspected of containing the target microparticle is analysed to detect, and optionally also quantify, the target microparticle in the sample fluid. In the method, a liquid possibly containing target microparticles is applied to the measuring channel of a microfluidic particle analysis device, and target microparticles are detected using electrical impedance spectroscopy (EIS), also known as impedance flow cytometry (IFC). The present method analyses impedance, and in the present context, the impedance between the electrodes, e.g. between the first electrode and the second electrode is considered to be the ratio of the complex representation of the voltage between the electrodes to the complex representation of the current flowing through the electrodes. As the applied voltage, in the present context, is from an alternating current, e.g. a sinusoidal signal, the impedance possesses a magnitude and a phase due to the linear relation between the voltage and the impedance. Detection of microparticles using EIS is well-described as a label-free technique, and the detection of particles in liquids using impedance can traditionally employ the amplitude, the phase or both of the amplitude and the phase. The present inventors have now surprisingly found that when a liquid containing target microparticles labelled with electrically conducting nanoparticles is applied to the measuring channel for analysis using EIS and the liquid has a conductivity of at least 5,000 μS/cm, e.g. in the range of 5,000 μS/cm to 50,000 μS/cm, microparticles labelled with the electrically conducting nanoparticles can be detected and differentiated from other microparticles, i.e. particles not labelled with electrically conducting nanoparticles, when using the phase as shown in FIG. 4 and FIG. 8. In contrast, when the impedance analysis does not include the phase, e.g. it includes only the amplitude, particles labelled with electrically conducting nanoparticles cannot be differentiated from non-labelled particles, as illustrated in FIG. 3. Moreover, when the conductivity is below 5,000 μS/cm, and the impedance analysis includes the phase, particles labelled with electrically conducting nanoparticles also cannot be differentiated from non-labelled particles (see FIG. 6). Thus, the invention provides a solution to the problem of how to detect a microparticle labelled with electrically conducting nanoparticles, e.g. metallic nanoparticles. The present method employs the phase, but the method may also employ both of the phase and the amplitude. The present inventors have furthermore surprisingly found that microparticles labelled with electrically conducting nanoparticles are detected without problems from measuring artefacts caused by the electrically conducting nanoparticles. Thereby, the quick response of EIS at microfluidic scale is efficiently coupled with the selectivity and specificity of labelling target microparticles and the sensitivity of EIS to provide an extremely sensitive detection of a target microparticle.

In the present method, an alternating current is applied to create an electric field in the operating space. In the present context, the alternating current may also be referred to as an AC or as an AC voltage, and the terms may be used interchangeably. In general, the alternating current, or AC voltage, has a sinusoidal variation in the voltage between the electrodes, although more complex variations of the voltage between the electrodes are also contemplated. Thus, in an example, a sinusoidal alternating current is employed in the method. It is however also contemplated that a direct current source may be employed. The applied alternating current is considered as a reference signal, and the phase of the monitored electrical signal is obtained, or ‘extracted’, from a comparison of the monitored electrical signal with the reference signal. The phase is typically expressed as a “phase angle” and in the present context the phase angle is expressed in the unit rad (or radian), e.g. as “phase [rad]”. The amplitude is typically expressed in the unit dB and in the present context as “amp [dB]”. The relevant frequency may also be indicated with the phase angle and the amplitude. Comparison of a reference signal and a monitored signal is well-known to the skilled person, and any method of extracting the phase may be used in the present method. Exemplary methods include using a phase detector or using in-phase and quadrature (I-Q) demodulation. In general terms, I-Q demodulation comprises two mixing operations: the monitored signal is mixed with the reference signal and the monitored signal is mixed with the reference signal delayed by 90 degrees. In this context, “mixing” is equivalent to the multiplication of two signals. When mixing two sinusoidal signals with the same frequency, the outcome signal may be low-pass filtered in order to remove the harmonics involved in the non-linear operation. As a result, the two resultant signals correspond to the in-phase and quadrature components of the monitored electrical signal, from which the amplitude and phase can be extracted. I-Q demodulation allows the simultaneous extraction of both the amplitude and the phase angle whereas other methods extract only the phase angle. When only the phase angle is extracted, the amplitude may be obtained using any supplementary method.

The target microparticle exposes an identification binding partner, and the method employs a recognition binding partner having a binding affinity for the identification binding partner. Thus, a complex between the identification binding partner and the recognition binding partner is formed in the method, and the complex between the identification binding partner and the recognition binding partner is labelled with electrically conducting nanoparticles. Thereby, the target microparticle exposing the identification binding partner is labelled with the electrically conducting nanoparticles. The identification binding partner may be considered to be complementary to the recognition binding partner, and the identification binding partner and the recognition binding partner may also be referred to as a binding partner pair, e.g. the complex may be referred to as a binding partner pair. The binding partner pair may be selected freely from the target microparticle of interest. For example, bacteria and other microorganisms typically expose one or more molecules that are unique to the microorganisms so that these molecules can be used to identify the microorganism. The identification may for example be made using a binding partner of complementary binding affinity to the molecule identifying the microorganism. The molecule identifying a microorganism may for example be a protein molecule, e.g. an antigen, or a sugar molecule, e.g. an oligosaccharide or a polysaccharide. The molecule, i.e. the identification binding partner, may be specific for a single species of microorganisms, e.g. bacteria, or it may be specific for a group, e.g. genera, of related microorganisms. For example, Gram positive and Gram negative bacteria can be differentiated by lipopolysaccharides on their surface, and the recognition binding partner, e.g. an antibody, may bind to lipopolysaccharides specific for Gram negative bacteria, which thus provide a group specific identification binding partner.

Thus, as used herein the term “binding partner” refers to either of two parts of a pair of molecules capable of binding specifically to each other, and it may therefore refer to either the antigen or the antibody of an antigen-antibody pair, to either the carbohydrate or the lectin of a carbohydrate-lectin pair, to either strand of DNA of a double stranded molecule of complementary DNA sequences etc. In general, suitable examples of pairs of binding partners include those formed by antigen-antibody interactions, compound-aptamer interactions, antibody-antibody interactions, protein-small molecule interactions, enzyme-substrate interactions, enzyme-substrate analogue interactions, lectin-carbohydrate interactions, drug-receptor interactions, streptavidin-biotin interactions, nucleic acid-nucleic acid interactions, nucleic acid-protein interactions etc. The term “antibody” refers to antibodies of any suitable isotype as well as antibody fragments containing the antigen binding specificity of a complete antibody. Antibodies may be of monoclonal or polyclonal origin, or they may be produced recombinantly.

The “identification binding partner” refers to the part of a pair of binding partners exposed by a target microparticle, so that the target microparticle can be identified by its identification binding partner when the recognition binding partner is bound to the identification binding partner. For example, the recognition binding partner may be immobilised on the electrically conducting nanoparticles. The identification binding partner may be any molecule existing naturally on the target microparticle, but it is also contemplated that any number of auxiliary binding partners may be employed in the method. For example, the target microparticle may be identified by binding an auxiliary binding partner to the identification binding partner and subsequently binding the recognition binding partner to the auxiliary binding partner bound to the identification binding partner. In the context of the disclosure, an “auxiliary binding partner” is thus a binding partner having an active binding site and a passive binding site. The active binding site of an auxiliary binding partner will bind specifically to the recognition binding partner or to the passive binding site of another auxiliary binding partner, and the passive binding site of the auxiliary binding partner will bind to the recognition binding partner or to the active binding site of the other auxiliary binding partner. Thus, for example, in the present method an auxiliary binding partner may be bound to the identification binding partner and to the recognition binding partner. Specifically, the active binding site of the auxiliary binding partner is bound to the identification binding partner, and the passive binding site of the auxiliary binding partner is bound to the recognition binding partner. Likewise, in the present method a first auxiliary binding partner may be bound to the identification binding partner, and a second auxiliary binding partner may be bound to the first auxiliary binding partner and to the recognition binding partner. Specifically, the active binding site of the first auxiliary binding partner is bound to the identification binding partner, the passive binding site of the first auxiliary binding partner is bound to the active binding site of the second auxiliary binding partner, and the passive binding site of the second auxiliary binding partner is bound to the recognition binding partner.

When an auxiliary binding partner is employed in the method, it is preferred that the auxiliary binding partner has two or more, e.g. a plurality, of passive binding sites, in particular a plurality of identical passive binding sites. In particular, the auxiliary binding partner may be defined in terms of the ratio between the number of passive binding sites to the number of active binding sites. For example, the auxiliary binding partner may have one active binding site and two or more passive binding sites. When the auxiliary binding partner has two or more, e.g. a plurality, of passive binding sites, in particular a plurality of identical passive binding sites, especially when the ratio between the number of passive binding sites to the number of active binding sites is two or more, the number of recognition binding partners bound to the target microparticle via the auxiliary binding partner may be increased by a factor corresponding to the number of passive binding sites relative to the number of active binding sites. Thus. when an auxiliary binding partner has a plurality, of passive binding sites, in particular a plurality of identical passive binding sites, the sensitivity of the present method is increased compared to when no auxiliary binding partner is used, e.g. when the recognition binding partner is bound directly to the identification binding partner. An exemplary auxiliary binding partner with a plurality of passive binding sites is an antibody. For example, auxiliary binding partner may be an antibody to the identification binding partner, and the recognition binding partner may be an antibody to the antibody serving as the auxiliary binding partner. A further exemplary auxiliary binding partner comprises a molecule having the active binding partner, which molecule is linked, e.g. covalently bound, to a polymeric molecule, e.g. an oligosaccharide or a polysaccharide, and which polymeric molecule is linked, e.g. covalently bound, to a plurality of molecules, e.g. identical molecules, having the passive binding partner.

In the present method, the complex between the identification binding partner and the recognition binding partner is labelled. Thus, the recognition binding partner may be immobilised on an electrically conducting nanoparticle, and the target microparticles may be labelled by mixing sample fluid with the recognition binding partner component containing the recognition binding partner immobilised on the electrically conducting nanoparticle.

For example, the method may comprise the steps of
providing a microfluidic particle analysis device comprising a measuring channel having a cross-sectional dimension in the range of 1 μm to 70 μm and a sensor system for detecting a particle, which comprises a first electrode and a second electrode defining an operating space between the first electrode and the second electrode, which first and second electrodes are in electrical connection via an electric circuit comprising an alternating current source and a device for monitoring an electrical signal from the first and/or the second electrode,
providing a sample fluid suspected of containing the target microparticle, which target microparticle exposes an identification binding partner,
providing a recognition binding component comprising a recognition binding partner immobilised on an electrically conducting nanoparticle,
mixing the recognition binding partner component with the sample fluid to provide an application suspension comprising target microparticles labelled with the electrically conducting nanoparticles via a complex of the recognition binding partner and the identification binding partner;
adjusting the conductivity of the sample fluid or the application suspension to be in the range of 5,000 μS/cm to 50,000 μS/cm, if the conductivity of the sample fluid or the application suspension is below 5,000 US/cm;
applying a flow of the application suspension to the measuring channel of the microfluidic particle analysis device; and
applying an alternating current from the current source to create an electric field in the operating space and monitoring an electrical signal between the first and the second electrode to detect target microparticle labelled with the electrically conducting nanoparticles from a phase of the electrical signal, and optionally also an amplitude of the electrical signal.

Thus, in an example of the method, the recognition binding partner is immobilised on electrically conducting nanoparticles. In the context of the present method, the term “immobilised” means that the recognition binding partner is bound to the electrically conducting nanoparticles using any approach as desired. For example, the recognition binding partner may be adsorbed to the electrically conducting nanoparticles, the recognition binding partner may be bound to the electrically conducting nanoparticles via ionic interactions, the recognition binding partner may be covalently bound to the electrically conducting nanoparticles, etc. Immobilisation of a binding partner to a nanoparticle is well-known to the skilled person, and any method to immobilise a binding partner to a nanoparticle may be used in the present method. For example, the electrically conducting nanoparticles may have an outer surface of a metal, e.g. gold or silver, and a protein based recognition binding partner, e.g. an antibody, may be bound to the metal, e.g. the gold, via —SH groups of corresponding amino acids. When the complex of the recognition binding partner and the identification binding partner is formed, the target microparticles are thus labelled with electrically conducting nanoparticles.

The electrically conducting nanoparticles comprise a recognition binding partner that has a binding affinity for the identification binding partner. In general, the identification binding partner and the recognition binding partner take part in the reaction in Reaction 1 to form a complex between the identification binding partner and the recognition binding partner. In Reaction 1, BP_{Identification} is the identification binding partner, BP_{Recognition} is the recognition binding partner, and is BP_{Complex} the complex formed between the identification binding partner and the recognition binding partner.

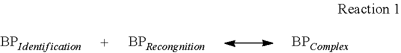

the binding affinity may be described in terms Equation 1:

$${K}_{A} = \frac{[ B {P}_{Complex} ]}{[ B {P}_{Identification} ] [ B {P}_{Recognition} ]} \qquad Equation 1$$

In Equation 1, K_{A} is the association constant, and [BP_{Comlex}], [BP_{Identification}] and [BP_{Recognition}] are the concentrations of the complex between the identification binding partner and the recognition binding partner, the concentration of unbound identification binding partner, and the concentration of unbound recognition binding partner, respectively at equilibrium. It is preferred that the affinity between the recognition binding partner and the identification binding partner, as expressed with the dissociation constant, K_{D}=K_{A}⁻¹, is in the range of 10⁻¹⁵ M to 10⁻⁵ M.

The present method uses a microfluidic particle analysis device for detecting the target microparticle in a fluid using electrical impedance spectroscopy (EIS). In the present context, EIS in a microfluidic particle analysis device may also be referred to as impedance flow cytometry (IFC), and a microfluidic particle analysis device having a measuring channel with electrodes configured to be used in the present method may be referred to as an impedance flow cytometer. In the present context the term “microfluidic” is intended to cover a range of sizes where the smallest dimension of channels is in the range from about 1 μm to about 1 mm, e.g. about 10 μm to about 200 μm, and in general the channels will not contain constrictions. It can generally be said that fluids in microfluidic fluidic systems will be flowing under laminar conditions, and fluidic systems with channels different from those defined above may well be described as “microfluidic” as long as fluids contained in the systems flow under laminar conditions.

The microfluidic particle analysis device comprises a measuring channel having a cross-sectional dimension in the range of 1 μm to 70 μm and a sensor system for detecting a particle, which comprises a first electrode and a second electrode defining an operating space between the first electrode and the second electrode. It is to be understood that a channel does not need to be fully enclosed by channel walls, and for example a channel, e.g. the measuring channel. may be open along a length axis to another channel, e.g. a larger channel. A corresponding device is disclosed in WO 2019/025613, where there is a main channel that along a vertex has an opening extending along a main flow direction, which opening is open to open to an analysis section, which corresponds to a measuring channel in the present context, but where the measuring channel is thus not fully enclosed by channel walls. The first electrode and the second electrode may also be referred to as a “particle detection system”. Thus, the particle detection system comprises a first electrode and a second electrode defining an operating space between the first electrode and the second electrode, which first and second electrodes are in electrical connection via an electric circuit comprising an alternating current source, and a device for monitoring an electrical signal from the first and/or the second electrode.

The measuring channel comprises a first electrode and a second electrode defining an operating space between the first electrode and the second electrode. The section of the measuring channel housing the electrodes may also be referred to as the analysis section. The first and the second electrode may be on the same surface of the analysis section, e.g. the first and the second electrode may be in a “coplanar” set-up, or the first and the second electrode may be positioned on opposite surfaces in the analysis section, e.g. the first and the second electrode may be in a “parallel overlap” set-up. When first and the second electrode are positioned on opposite surfaces in the analysis section, the set-up may also be referred to as “front facing”. When the two electrodes are coplanar the operating space is parallel to the direction of the flow in the analysis section, and the operating space is the distance between the electrodes, i.e. from the edge of the first electrode to the edge of the second electrode. The operating space of coplanar electrodes may be in the range of 1 μm to 70 μm, e.g. 1 μm to 50 μm, or 1 μm to 20 μm.

When the two electrodes are in a parallel overlap set-up the operating space is equal to the analytical distance between the first and the second surface with due consideration of the thickness of the electrodes. In general, the electrodes are flush with the surface, although the electrodes may rise up to about 1 μm, typically up to about 500 nm, e.g. 200 nm, from the surface, which is considered not to impact the behaviour of the liquid in the microfluidic particle analysis device so that no effects are caused by such electrodes. The first and the second electrode are generally of the same size, e.g. with superficial dimensions in the range of 1 μm to 100 μm, e.g. 5 μm to 50 μm, although the first and the second electrode may also have different sizes. The electrodes may be of any conducting material but are generally metallic, e.g. prepared from titanium, gold, nickel, copper, iridium, platinum, palladium, or combinations and alloys thereof.

The electrodes are in electrical connection via an electric circuit comprising the alternating current source and a device for monitoring an electrical signal. The electrical circuit may comprise conductors, which are integrated with the microfluidic particle analysis device, e.g. in the substrate of the microfluidic particle analysis device. The alternating current source may be chosen as appropriate, and an alternating current source may provide frequencies in the kHz to MHz range, e.g. from 100 kHz to 100 MHZ, or 1 MHZ to 20 MHz. The voltage between the first and the second electrode will typically be in the range of 0.1 V to 20 V, e.g. 0.5 V to 5 V. The device for monitoring an electrical signal may comprise a processing device for analysing a signal recorded from the electrodes. The device for monitoring an electrical signal may further comprise an output device for displaying or transferring data from the device for monitoring the electrical signal. A device for transferring data may operate using any wireless or wired data transmission protocol.

The alternating current may be provided at a single frequency, or the method may employ at least two, e.g. two or more, different frequencies. For example, the method may use a first frequency in the range of 100 kHz to 100 MHz, e.g. 1 MHz to 20 MHz, and a second frequency in the range of 100 kHz to 100 MHz, e.g. 1 MHz to 20 MHz, which second frequency is different from the first frequency. When two different frequencies are employed, particles labelled with electrically conducting nanoparticles, e.g. metallic nanoparticles, can more easily be differentiated from non-labelled particles than when using only a single frequency.

The measuring channel comprises electrodes, and in the present context the electrodes can be referred to as sets of electrodes. Each set of electrodes has a first electrode and a second electrode. The measuring channel may comprise an upstream set of electrodes and a downstream set of electrodes. The upstream set of electrodes may also be referred to as an onset set of electrodes, and the downstream set of electrodes may also be referred to as a balance set of electrodes. In use, a voltage is applied to the first electrode and a current is measured at the second electrode. The first electrode may also be referred to as the “excitation electrode”, and the second electrode may also be referred to as the “reference electrode”. The measured current is recorded, e.g. continuously, at a predetermined sample rate. When a liquid without any particles passes the electrodes, e.g. the operating space, the reference electrode will provide a “base signal”, and when a particle, such as a biological cell, e.g. a bacterium, passes the operating space the signal will change. The upstream set of electrodes and the downstream set of electrodes, when both an upstream set of electrodes and a downstream set of electrodes are used, are in electrical connection via an electric circuit. It is preferred that the circuit includes both of the upstream set of electrodes and the downstream set of electrodes, so that when a voltage is applied to the first electrodes, the same voltage is applied to the first electrodes of the upstream set of electrodes and the downstream set of electrodes. Thereby, a differential electrical signal between the upstream set of electrodes and the downstream set of electrodes can be recorded. In an example, the upstream set of electrodes and the downstream set of electrodes share a single first electrode, i.e. the upstream set of electrodes and the downstream set of electrodes share an excitation electrode. The shared excitation electrode is preferably arranged between the second electrodes, i.e. the reference electrodes, of the upstream set of electrodes and the downstream set of electrodes relative to the direction of flow in the measuring channel. In the present context, the term “differential signal” or “differential electrical signal” means the signal obtained by subtracting the signal of the downstream set of electrodes from the signal of the upstream set of electrodes. When the same voltage is applied to the upstream set of electrodes and the downstream set of electrodes, and no particle is detected by the electrodes, the differential signal is zero, except for random noise. Using a differential electrical signal allows that common mode noise can be recognised, and that the dynamic range at the receiver is increased and, correspondingly, a particle can be detected with greater sensitivity.

In a specific embodiment the electrodes are arranged in a coplanar set-up, and the particle detection system comprises an excitation electrode located between two reference electrodes. The measurement electrodes comprise a first reference electrode upstream of the excitation electrode and a second reference electrode downstream of the excitation electrode. In this embodiment the operating space is divided into an onset operating space between the first reference electrode and the excitation electrode and a balance operating space between the excitation electrode and the second reference electrode. In use, a voltage is applied to the excitation electrode and a current is measured at the two reference electrodes. A particle passing through the operating space will first encounter the onset operating space where its presence will be recorded by a change in the signal between the excitation electrode and the first reference electrode. When the particle is in the onset operating space no change in the signal will be recorded between the excitation electrode and the second reference electrode, but when the particle reaches the balance operating space its presence will be recorded by a change in the signal between the excitation electrode and the second reference electrode, whereas no change in the signal will be recorded between the excitation electrode and the first reference electrode. This allows that the same particle is recorded twice by the electrode set-up, and thereby the velocity of a particle can be estimated. Measurement of the particle velocity allows that the overall flow velocity, e.g. the linear flow velocity, of the liquid in the measuring channel is estimated. Thus, this embodiment allows that the flow rate through the microfluidic particle analysis device is estimated. Knowledge of the fluid velocity in the measuring channel further provides a better estimate of the concentration of particles in the liquid than can be recorded when only a single reference electrode is employed, since the signals can be correlated with the estimated fluid velocity. This same effect can be obtained when the particle detection system comprises two or more sets of electrodes arranged in a parallel overlap set-up, wherein a first, i.e. upstream, set of electrodes define an onset operating space and a second, i.e. downstream set of electrodes define a balance operating space. In both embodiments the size of the onset operating space and the balance operating space may be the same, or the sizes may differ from each other. When two sets of electrodes in a parallel overlap set-up are employed the distance between the two sets of electrodes will typically be in the range of 5 μm to 50 μm, e.g. 10 μm to 20 μm.

The particle detection system is located in a measuring channel. The microfluidic particle analysis device may comprise no other channels than the measuring channel, or the microfluidic particle analysis device may comprise any number and type of additional channels. For example, the microfluidic particle analysis device may comprise a bypass-channel as disclosed in WO 2016/116535, or the measuring channel may be open to a main channel as disclosed in WO 2019/025613. Both of WO 2016/116535 and WO 2019/025613 are hereby incorporated by reference.

In a specific example, the microfluidic particle analysis device comprises an inlet in fluid communication via a main channel defining a main flow direction with an inlet manifold providing parallel fluid communication with
a bypass channel of hydrodynamic resistance R_{bypass}, and
a measuring channel of hydrodynamic resistance R_{measuring}, the measuring channel having a cross-sectional dimension in the range of 1 μm to 50 μm and further having a sensor system for detecting a particle,

wherein a flow distribution parameter X_{measuring}=R_{measuring}⁻¹(R_{measuring}⁻¹+R_{bypass}⁻¹)⁻¹ is in the range of 10⁻⁶ to 0.25, wherein the angle of the measuring channel relative to the main flow direction is in the range of 0° to 60°, and wherein the angle of the bypass channel relative to the main flow direction is in the range of 0° to 60°, and the microfluidic particle analysis device further comprises an outlet in fluid communication with the bypass channel and the measuring channel. The particle detection system is located in the measuring channel.

In another specific example, the microfluidic particle analysis device comprises an inlet and an outlet in fluid communication via a main channel defining a main flow direction from an inlet end to an outlet end,
the main channel being defined by a main channel wall extending from the inlet end to the outlet end and having a first cross-sectional dimension in the range of 20 μm to 120 μm and a second cross-sectional dimension of at least 100 μm, which main channel has a first vertex opposite a second vertex, which vertices are located opposite each other in the second cross-sectional dimension,
the main channel wall at the first vertex and/or the second vertex having an opening extending along the main flow direction, and
the main channel wall along the opening being open to an analysis section having a first surface opposite a second surface at an analytical distance in the range of 5 μm to 50 μm or in the range of 5 μm to 70 μm, and a sensor system for detecting a particle. In this example, the main channel is the measuring channel, and the particle detection system is thus located in the main channel.

In the present method, a sample fluid suspected of containing the target microparticle is provided. The target microparticle may be any microparticle typically found together with other microparticles. In the context of the present disclosure, any microparticle that can pass through the measuring channel, the microparticle may be analysed in the present method. For example, a microparticle may be a particle having a size in the range of 0.5 μm to 50 μm, although the microparticle may also be smaller than 0.5 μm or larger than 50 μm. Exemplary target microparticles comprise specific bacteria, e.g. a pathogenic bacterium, in a population of other bacteria and microorganisms and possibly also further microparticles not being of biological origin. Correspondingly, the sample fluid may be any liquid where a target microparticle can exist. Exemplary sample fluids include water, e.g. drinking water, water from the sea or a lake, bodily fluids, such as blood, saliva or semen, other biological fluids, such as milk, fluids for food applications, e.g. beer, wine, soft drinks, or industrial liquids, such as fermentation broths.

It is also contemplated that the target microparticle may be smaller than 0.5 μm and that by labelling the target microparticle with electrically conducting nanoparticles, e.g. gold or silver nanoparticles, individual target microparticle may be cross-linked by the electrically conducting nanoparticles to form agglutinated microparticles large enough for detection in the present method. For example, an electrically conducting nanoparticle with an immobilised recognition binding partner typically has a plurality of immobilised recognition binding partner molecules, which can cross-link individual target microparticles having a plurality of identification binding sites. For example, the target microparticle may be a virus particle, e.g. a virus particle having a size in the range of 20 nm to 500 nm.

According to the present method, the conductivity is in in the range of 5,000 μS/cm to 50,000 μS/cm, e.g. to be in the range of 8,000 μS/cm to 50,000 μS/cm, e.g. in the range of 10,000 μS/cm to 50,000 μS/cm, such as 15,000 μS/cm to 50,000 μS/cm, when a flow of the liquid is applied to the measuring channel of the microfluidic particle analysis device. If the sample fluid has a conductivity below 5,000 μS/cm, the conductivity of the sample fluid is normally increased to bring the conductivity within the range of 5,000 μS/cm to 50,000 μS/cm. The conductivity may be adjusted as desired. In particular, salt, e.g. NaCl, may be added to the sample fluid. Salt may be added in a dry form or as a solution, in particular a concentrated solution. Regardless of how the salt is added, the change in volume of the sample fluid due to the addition of the salt may be monitored. The conductivity may be adjusted for the sample fluid or for the application suspension, and the time of the adjustment does not affect detection of the labelled target microparticles.

In the present method, electrically conducting nanoparticles comprising a recognition binding partner having a binding affinity for the identification binding partner are provided. The electrically conducting nanoparticles may be in any form as desired. It is preferred that the electrically conducting nanoparticles are provided as a suspension in an aqueous liquid. The concentration of the electrically conducting nanoparticles in the aqueous liquid is typically in the range of 103 ml⁻¹ to 109 ml⁻¹, e.g. 105 ml⁻¹ to 108 ml⁻¹ and the ratio between the aqueous liquid and the sample fluid is typically in the range of 1:1 to 1:1000, e.g. 1:5 to 1:100.

In an example, the electrically conducting nanoparticles are provided as a suspension in an aqueous liquid, which has a high conductivity so that by mixing the aqueous liquid containing the electrically conducting nanoparticles with the sample fluid, the resulting application suspension containing the electrically conducting nanoparticles reaches a conductivity in the range of 5,000 μS/cm to 50,000 μS/cm. For example, the ratio between the aqueous liquid and the sample fluid is typically in the range of 1:1 to 1:1000, e.g. 1:5 to 1:100, and the conductivity of the aqueous liquid relative to the sample fluid is selected according to the conductivity of the sample fluid and the intended volumetric ratio between aqueous liquid and the sample fluid.

The application suspension of the electrically conducting nanoparticles is applied to the measuring channel of the microfluidic particle analysis device. Thus, in use a flow of liquid is led through the microfluidic particle analysis device, e.g. from an inlet to an outlet, and the liquid flowing through the microfluidic particle analysis device is analysed for a content of particles, e.g. the particles are “detected”. The microfluidic particle analysis device can also be referred to as a flow system. The flow rate of the application suspension through the microfluidic particle analysis device, in particular the measuring channel, is generally not limited, and the detection of target microparticles does not depend on the flow rate. For example, the linear flow velocity in the measuring channel may be in the range of 1 mm/s to 10 m/s, e.g. 10 mm/s to 100 mm/s, or 100 mm/s to 1,000 mm/s. The flow rate in the measuring channel may also be expressed as a volumetric flow, and the volumetric flow rate of the microfluidic particle analysis device may be in the range of 0.1 μl/min to 10 ml/min, e.g. 0.5 μl/min to 2 ml/min or 1 μl/min to 500 μl/min, e.g. 5 μl/min to 100 μl/min.

When detection of particles is performed over time the detection may also be referred to as “monitoring” the particles, e.g. the content of particles in a liquid may be measured. A flow system may be operated continuously or in batch mode. In the present context, the term “continuous” means that a flow is applied to the measuring channel over an extended period of time, and when a target microparticle is detected the detection is labelled with the point in time relative to the flow applied to the measuring channel. Detection of a target microparticle in the continuous mode may also be quantified relative to the volume of sample fluid flowing through the measuring channel, e.g. based on the flow rate. For certain applications, e.g. monitoring of streams of liquid, such as drinking water or a process stream containing production cells, continuous flow is advantageous over batch wise analysis since a positive detection result can be obtained faster than when samples need to be extracted and analysed, e.g. the time between sampling is reduced to zero. In the present context, the term “batch mode” means that a volume, in particular a defined volume, of sample fluid is analysed in the present method. Operation in batch mode is preferred for detection of pathogenic bacteria or microorganisms in small samples, e.g. blood samples.

The microfluidic particle analysis device may be a flow system where a flow of liquid enters the inlet and leaves the microfluidic particle analysis device via the outlet. Thus, the inlet and the outlet may define a direction of the flow in the microfluidic particle analysis device, and in this context elements of the microfluidic particle analysis device may be “upstream” or “downstream” relative to each other with respect to the direction of flow.

The microfluidic particle analysis device comprises at least a measuring channel, but the microfluidic particle analysis device may also comprise other channels. In the context of the invention a channel may have any cross-sectional shape, e.g. the channel may be square, rectangular, round, etc. The microfluidic particle analysis device is not limited to channels of the same cross-sectional shape, and the cross-sectional shape of a single channel may vary over the length of the channel.

The microfluidic particle analysis device may comprise a pump, e.g. an external pump, for pushing liquid through the microfluidic particle analysis device via the inlet, and the microfluidic particle analysis device may also comprise an auxiliary pump, e.g. for aspirating liquid via the outlet. The pump may be any pump appropriate for the specific task, and exemplary pumps are a piston pump, a syringe pump, a peristaltic pump, a membrane pump, a diaphragm pump, a gear pump, a microannular gear pump, or any other appropriate type of pump.

In the present method, electrically conducting nanoparticles are employed to label microparticles of interest. In the context of the present disclosure a “nanoparticle” is a particle with a size in the range of 0.1 nm to 1000 nm. However, it is preferred that the electrically conducting nanoparticles have a size in the ranges of 1 nm to 100 nm, or 3 nm to 50 nm.

In general, any part of the electrically conducting nanoparticles may provide electrical conductivity to the electrically conducting nanoparticles. In particular, the current is conducted in the sample fluid or the suspension of the electrically conducting nanoparticles and through the electrically conducting part or parts of the electrically conducting nanoparticles. For example, the electrically conducting nanoparticles may be of an electrically conducting material, or they may comprise a core or a coating of an electrically conducting material. When the electrically conducting nanoparticles comprise a coating of an electrically conducting material, the electrically conducting nanoparticles typically have a core of another material, e.g. an electrically non-conducting material, and the other material may be fully or partly coated with the electrically conducting. The electrically conducting material is typically a metal, and noble metals, e.g. gold, silver or platinum, are preferred metals.

The electrically conducting nanoparticles are generally electrically conducting by having at least a coating or a core of a metal, although it is also contemplated that other electrically conducting materials may be used. For example, graphite is electrically conducting and the electrically conducting nanoparticles may be or comprise a core of graphite.

In an example, the electrically conducting nanoparticles comprise a core of an electrically non-conducting material and a coat of an electrically conducting metal, e.g. gold or silver. The electrically non-conducting material of the core may be selected to provide a specific functionality to the electrically conducting nanoparticles. For example, the electrically non-conducting material may be a glass or polymer of a lower density than the metal coating the core of the electrically conducting nanoparticles so that the density of the electrically conducting nanoparticles can be lowered compared to electrically conducting nanoparticles made of the corresponding metal.

In another example, the core of an electrically non-conducting material is a superparamagnetic nanoparticle. Superparamagnetic particles are well-known to the skilled person, and any superparamagnetic material may be selected for the core. For example, the superparamagnetic nanoparticle may be a nanoparticle representing a single magnetic domain, e.g. a nanoparticle of a magnetic iron oxide, e.g. magnetite, and having a size in the range of 3 nm to 50 nm. The superparamagnetic nanoparticle may also be a cluster of several single magnetic domain nanoparticles.

When the electrically conducting nanoparticle comprises superparamagnetic core, the superparamagnetic nature of the electrically conducting nanoparticle allows that the electrically conducting nanoparticle is manipulated using a magnetic field. For example, the microfluidic particle analysis device may comprise a section with a magnetic capturing area where a superparamagnetic nanoparticle contained in a sample fluid can be immobilised. Superparamagnetic nanoparticles immobilised in the magnetic capturing area may be subjected to further analyses while immobilised, or the superparamagnetic nanoparticles may be transferred from the magnetic capturing area to another site, e.g. a site outside of the microfluidic particle analysis, for analysis. A magnetic capturing area may comprise a channel having a smallest cross-sectional dimension in the range of 10 μm to 1000 μm and on or more pins, e.g. pins of a diameter in the range of 1 μm to 1000 μm, of a high magnetic permeability, e.g. an alloy of 80% nickel and 20% iron (“permalloy”) or the like, arranged between a first magnetic pole opposite a second magnetic pole across the channel, e.g. the pins may be at an angle in the range of 45° to 135° to a line between the first magnetic pole and the second magnetic pole.

Any embodiment of the invention may be used in any aspect of the invention, and any advantage for a specific embodiment applies equally when an embodiment is used in a specific aspect.

### BRIEF DESCRIPTION OF THE DRAWINGS

In the following the invention will be explained in greater detail with the aid of an example and with reference to the schematic drawings, in which

FIG. 1 illustrates a parallel overlap electrode layout in a measuring channel in a device for the present method;

FIG. 2 illustrates a co-planar electrode layout in a measuring channel in a device for the present method;

FIG. 3 shows plot of counts vs. amplitude for Gram-negative bacteria, Gram-positive bacteria, polystyrene particles, borosilicate beads and silver coated borosilicate beads at 16,000 μS/cm;

FIG. 4 shows plot of counts vs. phase angle for Gram-negative bacteria, Gram-positive bacteria, polystyrene particles, borosilicate beads and silver coated borosilicate beads at 16,000 μS/cm;

FIG. 5 shows a population plot with Gram-negative bacteria, Gram-positive bacteria and polystyrene particles at 800 μS/cm;

FIG. 6 shows a population plot with Gram-negative bacteria, Gram-positive bacteria and silver coated borosilicate beads at 800 μS/cm;

FIG. 7 shows a population plot with Gram-negative bacteria, Gram-positive bacteria and polystyrene particles at 16,000 μS/cm;

FIG. 8 shows a population plot with Gram-negative bacteria, Gram-positive bacteria, polystyrene particles, borosilicate beads and silver coated borosilicate beads at 16,000 μS/cm;

FIG. 9 shows a population plot with Gram-negative bacteria, Gram-positive bacteria, borosilicate beads and silver coated borosilicate beads at 4,000 μS/cm;

FIG. 10 shows a population plot with Gram-negative bacteria, Gram-positive bacteria, polystyrene particles and silver coated borosilicate beads at 8,000 μS/cm;

FIG. 11 shows a population plot with Gram-negative bacteria, Gram-positive bacteria, polystyrene particles and silver coated borosilicate beads at 12,000 μS/cm.

The invention is not limited to the embodiments illustrated in the drawings. Accordingly, it should be understood that where features mentioned in the appended claims are followed by reference signs, such signs are included solely for the purpose of enhancing the intelligibility of the claims and are in no way limiting on the scope of the claims

### DETAILED DESCRIPTION

The present invention relates to a method of detecting a target microparticle 5 in a fluid in a microfluidic particle analysis device 1 comprising a measuring channel 2 with a first electrode 4 and a second electrode 4 defining an operating space 3 between and being in electrical connection with an electric current source and a device for monitoring an electrical signal from the first and/or the second electrode 4. An appropriate microfluidic particle analysis device is the microfluidic particle analysis device 1 known as BactoBox (from SBT Instruments A/S, Herlev, Denmark), but the microfluidic particle analysis devices disclosed in WO 2016/116535 and WO 2019/025613 are likewise appropriate.

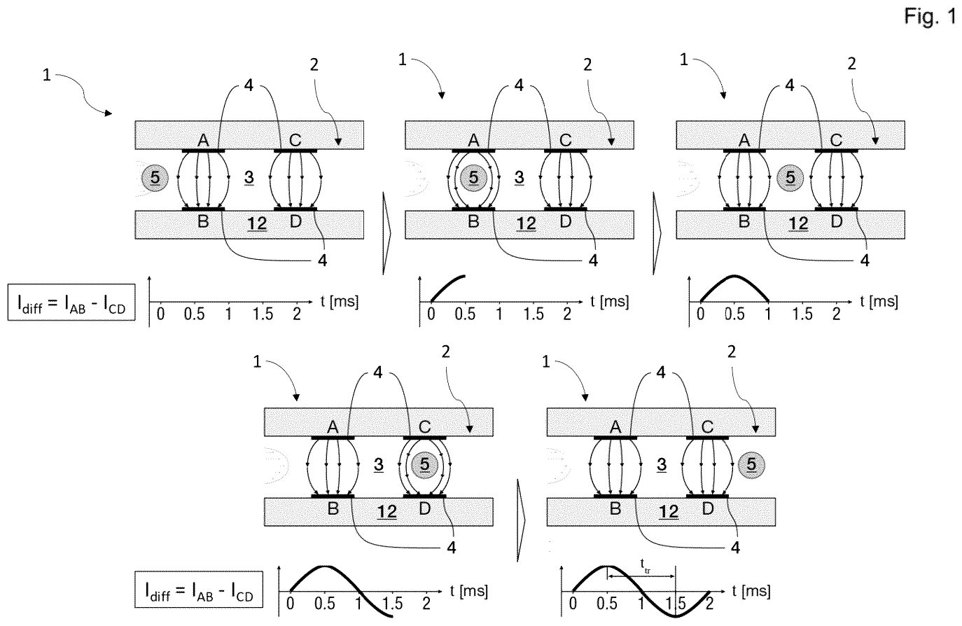

A section of a microfluidic particle analysis device 1 for use in the method, where the electrodes 4 are in a parallel overlap set-up, is illustrated in FIG. 1. FIG. 1 shows the operating space 3 of the measuring channel, where the electrodes 4 are located. The electrodes 4 are formed on a substrate 12. In FIG. 1, the electrodes 4 are specified with an additional label, “A” to “D”. In this example, the microfluidic particle analysis device 1 comprises a first excitation electrode A facing an opposite first reference electrode B. Additionally, a second excitation electrode C opposite a second reference electrode D is located downstream of the first excitation electrode A and the first reference electrode B. A voltage, e.g. as an alternating current, is applied to the excitation electrodes A, C and a current is measured at the two reference electrodes B,D. The signals from the two reference electrodes B,D are subtracted (I_{diff}=I_{AB}−I_{CD}) in order to obtain a characteristic transition signal as illustrated in FIG. 1. When no particle 5 is present between the electrodes A-B or C-D the measured current is equal at electrode A and B (I_{AB}=I_{CD}), and the differential signal is therefore zero (I_{diff}=0). As the particle 5 moves into the volume between the upstream excitation electrode A and its reference electrode B, i.e. the operating space 3, the signal measured on the upstream reference electrode B changes. The signal on the downstream reference electrode D will, however, not change and the differential current will be different from zero (I_{diff}≠0). The maximum differential current is measured when the particle 5 is positioned exactly between the upstream excitation electrode A and its reference electrode B. When the particle is exactly in-between the A and B electrodes and the C and D electrodes in the flow stream direction, the measured signals will again be equal (I_{diff}=0). The minimum differential current is measured when the particle is positioned between excitation electrode C and its reference electrode D.

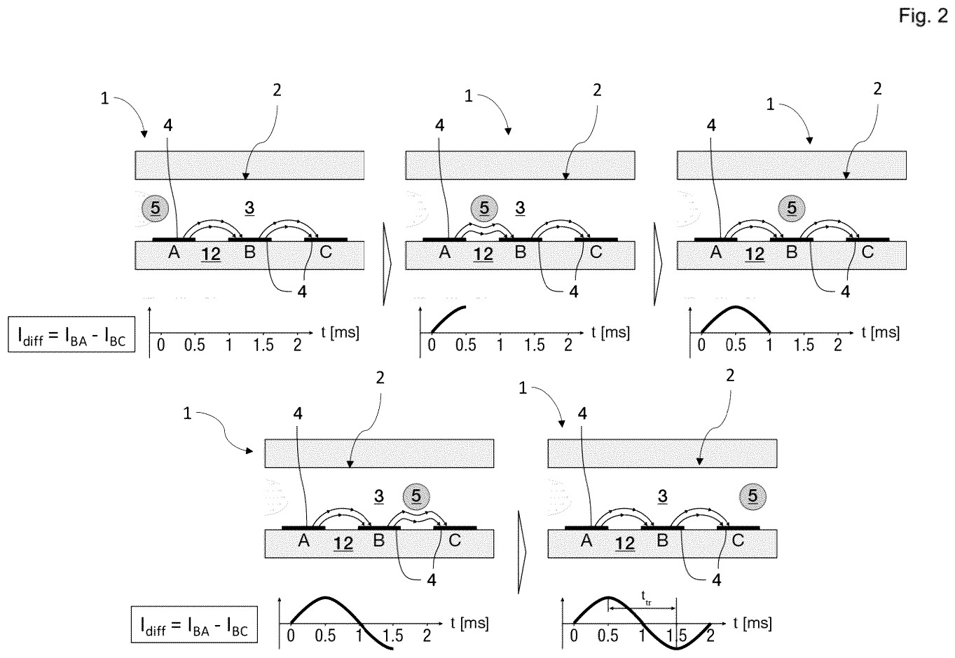

The section of another microfluidic particle analysis device 1 for use in the method is illustrated in FIG. 2. FIG. 2 shows the operating space 3 of the measuring channel, where the electrodes 4 are located. The electrodes 4 are formed on a substrate 12. In FIG. 2, the electrodes 4 are arranged in a coplanar set-up, and the microfluidic particle analysis device 1 comprises an excitation electrode B located between two reference electrodes A, C. A voltage is applied to the excitation electrode B and a current is measured at the two reference electrodes A,C. The signals from the two reference electrodes A, C are subtracted (I_{diff}=I_{BA}−I_{BC}) in order to obtain a characteristic transition signal as illustrated in FIG. 2. When no particle 5 is present between the electrodes 4 the measured current is equal at electrode A and C (I_{BA}=I_{BC}), and the differential signal is therefore zero (I_{diff}=0). As the particle 5 moves into the volume between the upstream reference electrode A and the excitation electrode B, i.e. the operating space 3, the signal measured on the upstream reference electrode A changes. The signal on the downstream reference electrode C will, however, not change and the differential current will be different from zero (I_{diff}≠0). The maximum differential current is measured when the particle 5 is positioned exactly between the upstream reference electrode A and the excitation electrode B. When the particle 5 is exactly above the centre of excitation electrode B, the measured signals will again be equal (I_{diff}=0). The minimum differential current is measured when the particle is positioned exactly between excitation electrode B and downstream reference electrode C.

The magnitude and shape of the transition signal at several frequencies is used to characterise the particle properties and sample features thus determining the type of particles 5 in the sample. Additionally, the transition signal can be used to determine the velocity with which the particle 5 moves across the electrodes 4, by considering the length the particle has moved and the time of the transition (tₜᵣ). The time can be determined directly from the transition signal by evaluating the time from the maximum peak to the minimum peak. The time of the transition (tₜᵣ) is indicated in FIG. 1 and FIG. 2. The distance travelled by the particle 5 is evaluated by considering two things. First of all, the width of the electrodes 4 and the distance between them, which are specific dimensions chosen during the design of the microfluidic particle analysis device 1 and are very well defined. Secondly, due to the microscopic dimensions of the measuring channel 2, the flow in the measuring channel 2 is laminar. This means that the particle 5 will stay in the same position in the measuring channel 2, e.g. the analysis section 3, during a transition, and will move in a straight line across the electrodes 4. Thus, by determining the time between the maximum and minimum differential current and the physical distance that the particle 5 has travelled, the exact velocity of the particle 5 can be calculated (see FIG. 1 and FIG. 2). By evaluating the flow velocity of the particles 5 and using the well-defined channel dimensions one can easily determine the flow rate in the analysis section 3, as the particles 5 will follow the flow in the analysis section 3 under any given condition applied.

The microfluidic particle analysis device 1 may be fabricated using any appropriate technology, but it is preferred that the microfluidic particle analysis device 1 is fabricated using cleanroom facilities due to the small critical dimensions of channels. The fabrication process may thus involve standard fabrication procedures such as an electrode lift-off process, photolithography and direct bonding, as are well-known to the skilled person.

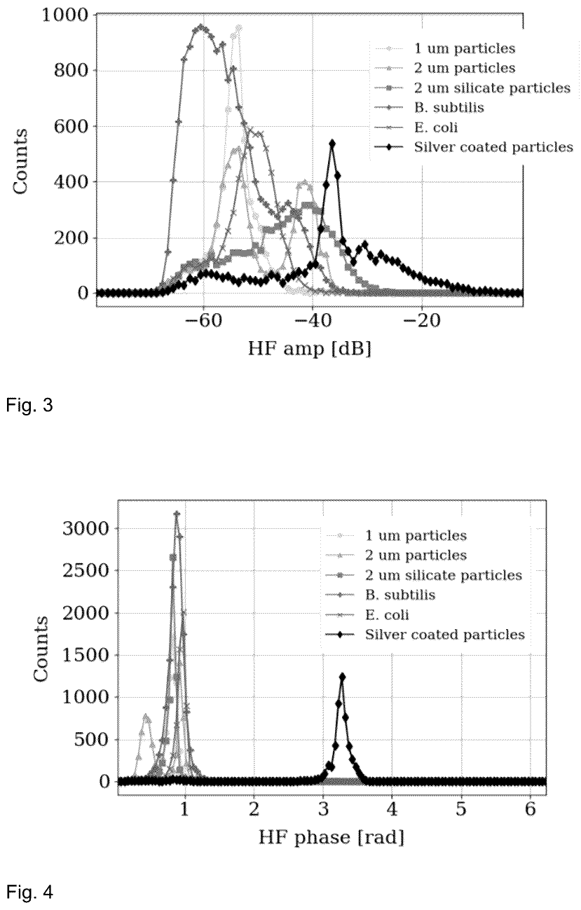

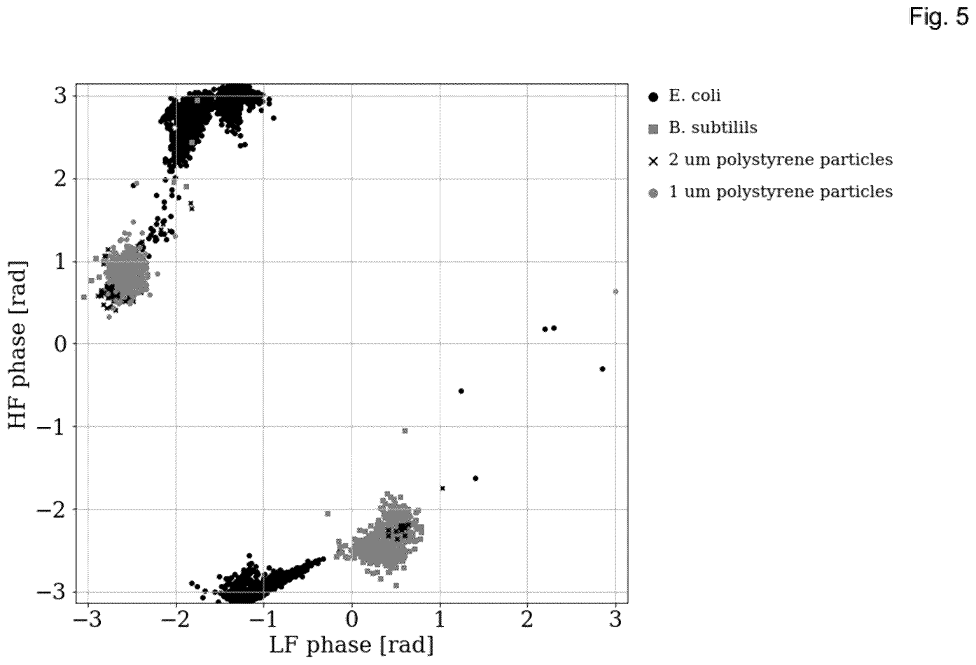

Electrical impedance spectroscopy (EIS) experiments were conducted in a BactoBox device as an exemplary microfluidic particle analysis device 1 to illustrate the present method, but the experiments can readily be performed in devices disclosed in WO 2016/116535 and WO 2019/025613. For the Examples below, different conductivity-scenarios were tested with the BactoBox at conductivities of 800 μS/cm, 4,000 μS/cm, 8,000 μS/cm, 12,000 μS/cm and 16,000 μS/cm, achieved by diluting Phosphate buffered saline (PBS) in Water For Injection (ultra pure water). The following particles were used: 1 μm polystyrene microspheres (Polysciences, cat: 07310-15), 2 μm polystyrene microspheres (Polysciences, cat: 19814-15), 2 μm borosilicate microspheres with an 85 nm silver coating (Cospheric, cat: SiO₂MS-AG-4.1) Escherichia coli ATCC 8739 ATCC and Bacillus subtilis subsp. spizizenii ATCC 6633. E. coli and B. subtilis were picked as a single colony from a plate and inoculated in 8 ml Brain Heart Infusion broth and grown overnight at 35° C., 300 rpm. Prior to being measured, the cultures and microspheres were diluted to between 500,000 to 1,000;000 cells/ml and microspheres/ml, respectively, in the different conductivity liquids. The measurements were performed using a single frequency (7 MHZ) or the measurements were performed at two frequencies of approximately 2 MHz and 7 MHz, and in all cases, the input signal was varied between 3V peak-to-peak for high conductivity samples to 15V peak-to-peak for low conductivity samples. Results were analysed and plotted using a custom-made software-program. Specifically, the I-Q demodulation and event detection were implemented in a Field Programmable Gate Array (FPGA) which is an embedded system that allows simultaneous software and hardware development. Every event was detected as a “count” and the posterior data analysis of the events was carried out in Python, although MATLAB (MathWorks Inc., Natick, MA, USA) may also be used in the data processing. Thus, the results were plotted as the number of counts against the amplitude (expressed as “HF amp (dB)”) in FIG. 3 or the number of counts against the phase angle (expressed as “HF phase [rad]”) in FIG. 4, or the results were plotted with the 2 MHz on the X-axis (where LF phase indicates “low” frequency phase for the 2 MHZ) and the 7 MHz on the Y-axis (where HF phase indicates “high” frequency phase for 7 MHZ) in FIG. 5 to FIG. 11. A single frequency can be used to differentiate the metal labelled particles from the non-labelled particles, but by using two frequencies and plotting the high frequency vs. the low frequency, differences in signals are visualised and thereby it is shown that certain particle types can be differentiated from each other.

### Example 1

Samples of Gram-negative E. coli bacteria, Gram-positive B. subtilis bacteria, polystyrene particles of 2 μm and 1 μm, respectively, 2 μm borosilicate beads and silver coated 2 μm borosilicate beads were suspended in PBS to provide a conductivity of about 16,000 μS/cm, and the samples were applied individually to a BactoBox microfluidic particle analysis. The amplitudes and the phase angles were extracted from the data, and recorded counts were plotted against the amplitudes in FIG. 3, and recorded counts were plotted against the phase angles in FIG. 4. The data are shown overlaid in FIG. 3 and FIG. 4.

As evident from FIG. 3, the silver coated particles did show a peak in counts differing slightly from the peaks of the other particle types, but the overlap in the curves for the particles show that the particles could not be differentiated by using only the amplitude. However, when the plot includes the phase data (FIG. 4), the silver coated particles clearly stand out from the other particle types and using the phase angle allows that the silver coated particles are clearly differentiated from the non-labelled particle types.

### Example 2

Samples of Gram-negative E. coli bacteria, Gram-positive B. subtilis bacteria and polystyrene particles of 2 μm and 1 μm, respectively were suspended in 5% PBS to provide a conductivity of about 800 μS/cm, and the samples were applied individually to the microfluidic particle analysis device. The samples were applied separately to the microfluidic particle analysis device, and the electrical signals were recorded and plotted in FIG. 5. In FIG. 5, E. coli are shown as black dots, B. subtilis as grey squares and particles of 2 μm and 1 μm as black crosses and grey dots, respectively. It is clear from FIG. 5 that the bacteria are positioned differently from the polystyrene particles, and from each other so that the Gram-negative E. coli bacteria, Gram-positive B. subtilis bacteria and polystyrene particles can be differentiated.

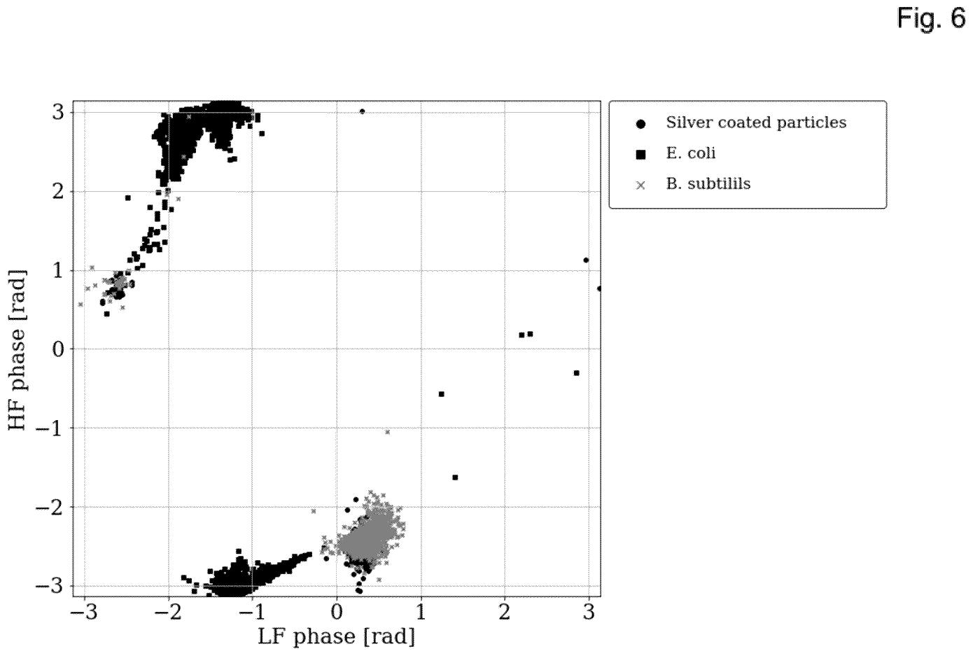

Samples of the silver coated 2 μm borosilicate beads were suspended in 5% PBS and applied individually to the microfluidic particle analysis device, and the electrical signals were recorded. The signal from the silver coated borosilicate beads is shown overlaid on the signals from the E. coli bacteria and the B. subtilis bacteria in FIG. 6. In FIG. 6, black dots represent the silicate particles with a silver coating, black squares represent E. coli and grey crosses represent B. subtilis. FIG. 6 shows that the signal of the silver coated borosilicate beads overlapped with the signal of the B. subtilis bacteria, and these could not be differentiated from each other.

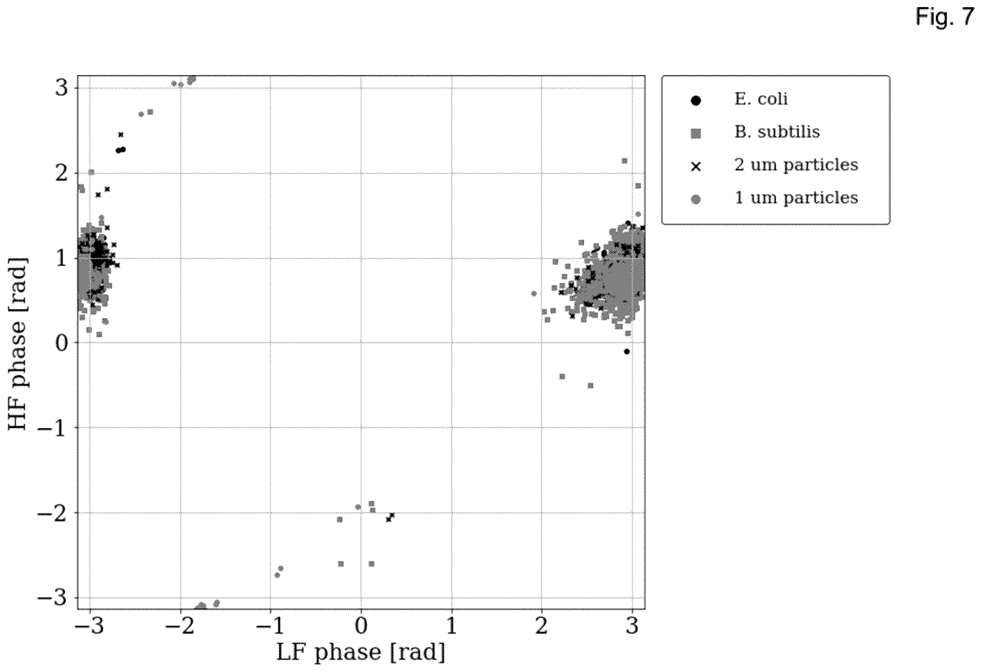

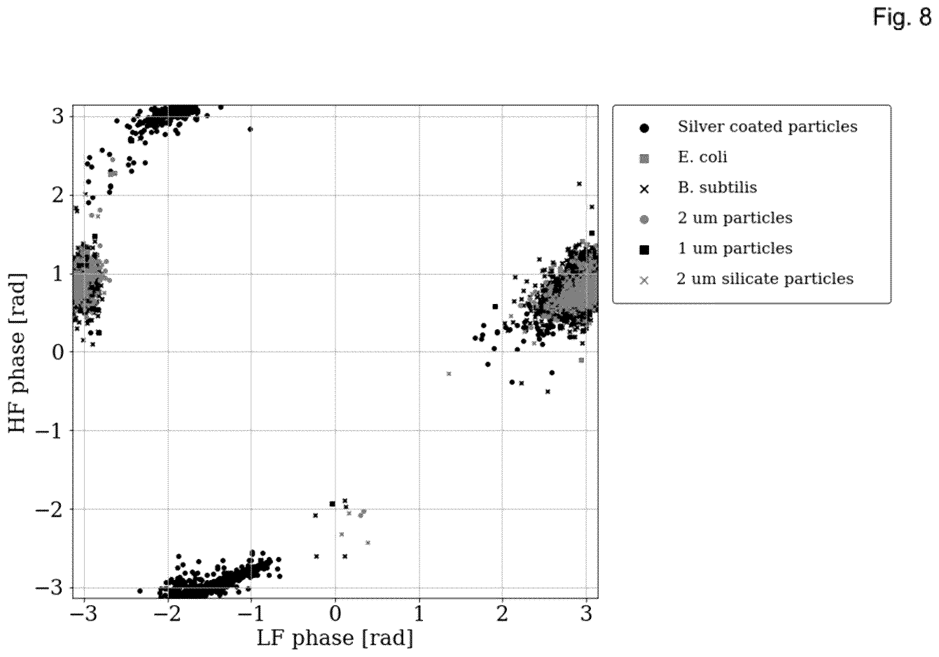

Samples of the 2 μm polystyrene particles, E. coli bacteria and B. subtilis bacteria were suspended in 100% PBS having a conductivity of about 16,000 μS/cm, and the suspensions were applied individually to the microfluidic particle analysis device, and the electrical signals were recorded. A population plot of the signals is shown overlaid in FIG. 7. In FIG. 7, black dots represent E. coli, grey squares represent B. subtilis, black crosses represent 2 μm polystyrene particles and grey dots represent 1 μm polystyrene particles. FIG. 7 illustrates that at the conductivity of PBS, neither of the particles, i.e. 2 μm polystyrene particles, E. coli bacteria and B. subtilis bacteria, could be differentiated as they all positioned equally in the population plot.

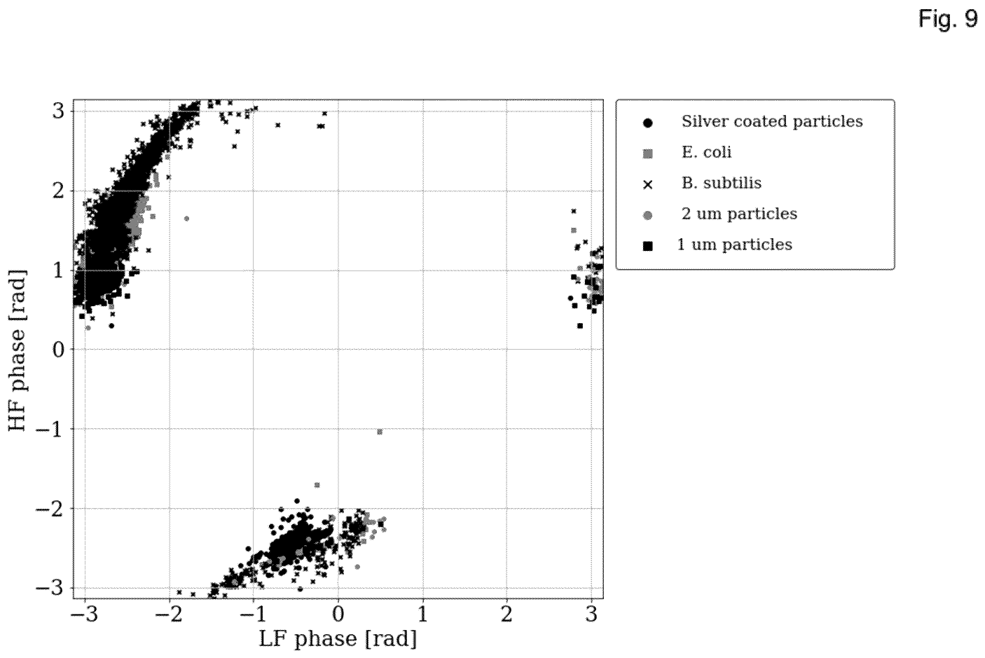

Samples of the silver coated 2 μm borosilicate beads, 2 μm borosilicate beads, polystyrene particles, E. coli bacteria and B. subtilis bacteria were suspended in 100% PBS having a conductivity of about 16,000 μS/cm and the suspensions were applied individually to the microfluidic particle analysis device, and the electrical signals were recorded. A population plot of the signals is shown overlaid in FIG. 8. In FIG. 8, black dots represent the 2 μm silicate particles with a silver coating, grey squares represent E. coli, black crosses represent B. subtilis, grey dots represent 2 μm polystyrene particles, black squares represent 1 μm polystyrene particles and grey crosses represent 2 μm silicate beads. FIG. 8 illustrates that upon silver coating the borosilicate beads and analysing the silver coated beads compared to uncoated polystyrene particles, the uncoated borosilicate beads, and bacteria, the silver coated beads were clearly differentiated from the other particles, which grouped together in the plots.

Thus, at a low conductivity of about 800 μS/cm, Gram-negative E. coli bacteria, Gram-positive B. subtilis bacteria and polystyrene particles could be readily differentiated from each other using EIS. However, at a high conductivity of about 16,000 μS/cm, Gram-negative E. coli bacteria, Gram-positive B. subtilis bacteria and polystyrene particles grouped together in the plots, and neither particle type could be differentiated. When a silver coating was used to label particles, in this case glass beads made of borosilicate, the silver coating allowed that the silver coated glass beads were clearly differentiated from the other particle types at the high conductivity, despite it that silver coated glass beads could not be differentiated from B. subtilis bacteria at low conductivity. The present inventors have thus surprisingly discovered that when silver coated particles are analysed in EIS at high conductivity, the silver coating allows detection of the silver coated particles compared to non-coated particles. Thereby, silver coating can be used to selectively label target microparticles for detection in EIS.

### Example 3

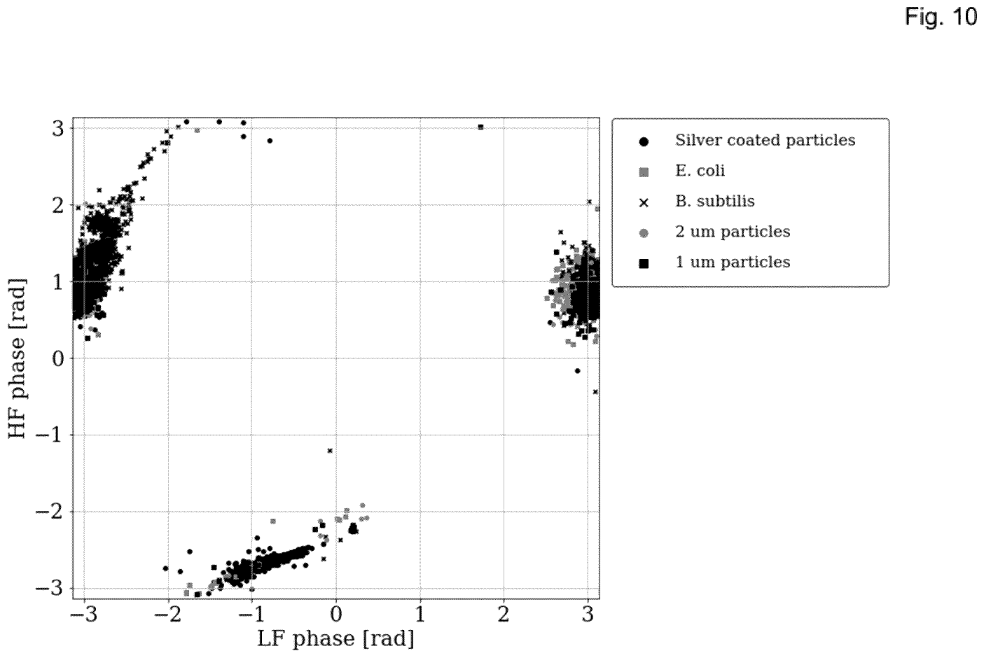

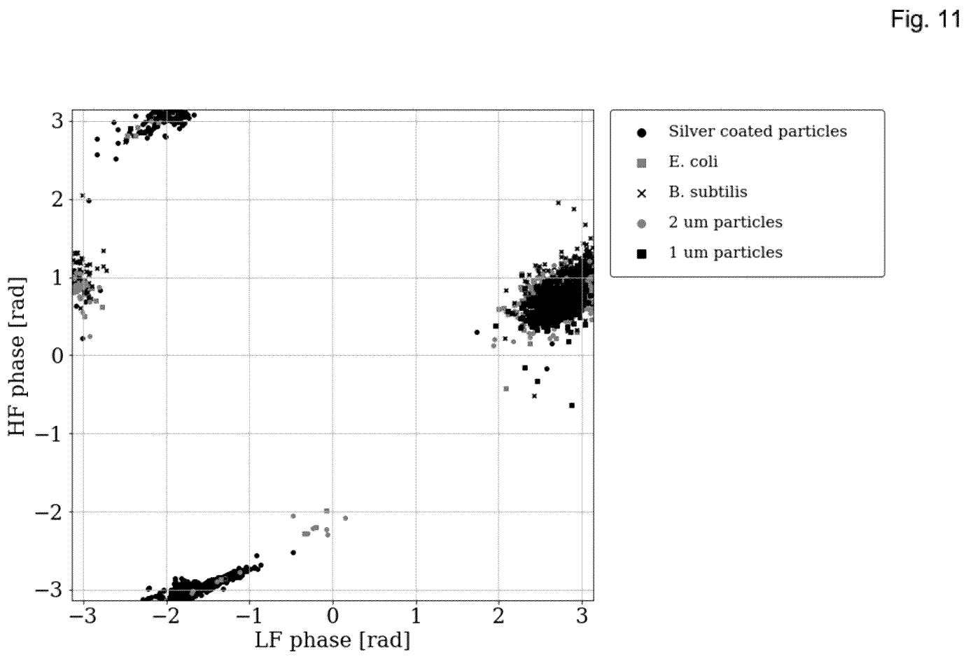

In order to analyse the effect of conductivity, experiments were conducted where the silver coated 2 μm borosilicate beads, polystyrene particles, E. coli bacteria, and B. subtilis bacteria were suspended in PBS at different concentrations, and the conductivity adjusted samples were applied individually to a BactoBox microfluidic particle analysis. Specifically, the samples were suspended in 25%, 50% and 75% PBS to provide conductivities of about 4,000 μS/cm, 8,000 μS/cm and 12,000 μS/cm, respectively. Population plots of measurements obtained at 4,000 μS/cm, are shown in FIG. 9, population plots of measurements obtained at 8,000 μS/cm are shown in FIG. 10, and population plots of measurements obtained at 12,000 μS/cm are shown in FIG. 11. In FIG. 9 to FIG. 11, black dots represent the 2 μm silicate particles with a silver coating, grey squares represent E. coli, black crosses represent B. subtilis, grey dots represent 2 μm polystyrene particles, and black squares represent 1 μm polystyrene particles.

FIG. 9 shows that at 4,000 μS/cm there is still some overlap between the B. subtilis bacteria and the silver coated glass beads, but as the conductivity increased, the signal of the silver coated glass beads became more clearly distinguished from the signals from the other particle types (FIG. 10 and FIG. 11). Thus, at a conductivity of 5,000 μS/cm, a silver coated particle can be differentiated from a particle, in particular a bacterium, not having a silver coating.

### REFERENCE SIGNS LIST

1 microfluidic particle analysis device
12 substrate
2 measuring channel
3 operating space
4 electrode
A first excitation electrode
B first reference electrode
C second excitation electrode
D second reference electrode
5 particle

1. A method of detecting a target microparticle in a fluid, the method comprising:
providing a microfluidic particle analysis device comprising a measuring channel having a cross-sectional dimension in the range of 1 μm to 70 μm and a sensor system for detecting a particle, which comprises a first electrode and a second electrode defining an operating space between the first electrode and the second electrode, which first electrode and which second electrode are in electrical connection via an electric circuit comprising an alternating current source and a device for monitoring an electrical signal from at least one of the first and/or the second electrode;
providing a sample fluid suspected of containing the target microparticle, which target microparticle exposes an identification binding partner;
providing a recognition binding component comprising a recognition binding partner having a binding affinity for the identification binding partner;
mixing the recognition binding partner component with the sample fluid to provide an application suspension comprising a complex of the recognition binding partner and the identification binding partner;
labelling the complex of the recognition binding partner and the identification binding partner with electrically conducting nanoparticles;
adjusting the conductivity of the sample fluid or the application suspension to be in the range of 5,000 μS/cm to 50,000 μS/cm, if the conductivity of the sample fluid or the application suspension is below 5,000 μs/cm;
applying a flow of the application suspension to the measuring channel of the microfluidic particle analysis device; and
applying an alternating current from the current source to create an electric field in the operating space and monitoring an electrical signal between the first electrode and the second electrode to detect target microparticles labelled with the electrically conducting nanoparticles from a phase of the electrical signal.

2. The method of detecting a target microparticle in a fluid according to claim 1, wherein the recognition binding component comprises the recognition binding partner immobilised on an electrically conducting nanoparticle.

3. The method of detecting a target microparticle in a fluid according to claim 1, wherein the electrically conducting nanoparticle is a silver nanoparticle or a gold nanoparticle.

4. The method of detecting a target microparticle in a fluid according to claim 1, wherein the electrically conducting nanoparticles comprises a core of an electrically non-conducting material and a coat of an electrically conducting metal.

5. The method of detecting a target microparticle in a fluid according to claim 4, wherein the electrically non-conducting core is a superparamagnetic nanoparticle.

6. The method of detecting a target microparticle in a fluid according to claim 1, wherein the dissociation constant between the recognition binding partner and the identification binding partner is in the range of 10⁻¹⁵ M to 10⁻⁵ M.

7. The method of detecting target microparticle in a fluid according to claim 6, wherein the recognition binding partner is selected from the group consisting of antigens, proteins, polypeptides, oligopeptides, polysaccharides, oligosaccharides, sugars, polynucleotides, and biotin.

8. The method of detecting a target microparticle in a fluid according to claim 1, wherein target microparticle is a pathogenic microorganism.

9. The method of detecting a target microparticle in a fluid according to claim 1, wherein the method further comprises detecting microparticles not labelled with the electrically conducting nanoparticles.

10. The method of detecting a target microparticle in a fluid according to claim 1, wherein the sensor system for detecting a particle comprises an upstream set of electrodes and a downstream set of electrodes with each set of electrodes having a first electrode and a second electrode, and the electrical signal between the first and the second electrode is a differential electrical signal.

11. The method of detecting a target microparticle in a fluid according to claim 1, wherein the alternating current is applied at two or more different frequencies.

12. The method of detecting a target microparticle in a fluid according to claim 1, wherein the flow of the application suspension is applied to the measuring channel continuously or in batch mode.

13. The method of detecting a target microparticle in a fluid according to claim 11, wherein a first frequency in the range of 100 kHz to 100 MHZ, and a second frequency in the range of 100 kHz to 100 MHz.

14. The method of detecting a target microparticle in a fluid according to claim 1, wherein the conductivity of the sample fluid or the application suspension is in the range of 8,000 μS/cm to 50,000 μS/cm.

15. The method of detecting a target microparticle in a fluid according to claim 1, wherein the conductivity of the sample fluid or the application suspension is in the range of 10,000 μS/cm to 50,000 μs/cm.

16. The method of detecting a target microparticle in a fluid according to claim 1, wherein the first and the second electrode are positioned on opposite surfaces in the analysis section.

17. The method of detecting a target microparticle in a fluid according to claim 1, wherein the analysis section has at least on surface, and the first and the second electrode are positioned on the same surface of the analysis section.

18. A method of detecting a target microparticle in a fluid, the method comprising:
providing a microfluidic particle analysis device comprising a measuring channel having a cross-sectional dimension in the range of 1 μm to 70 μm and a sensor system for detecting a particle, which comprises a first electrode and a second electrode defining an operating space between the first electrode and the second electrode, which first electrode and second electrode are in electrical connection via an electric circuit comprising an alternating current source and a device for monitoring an electrical signal from at least one of the first and the second electrode,
providing a sample fluid suspected of containing the target microparticle, which target microparticle exposes an identification binding partner,
providing a recognition binding component comprising a recognition binding partner immobilised on an electrically conducting nanoparticle,
mixing the recognition binding partner component with the sample fluid to provide an application suspension comprising target microparticles labelled with the electrically conducting nanoparticles via a complex of the recognition binding partner and the identification binding partner;
adjusting the conductivity of the sample fluid or the application suspension to be in the range of 5,000 μS/cm to 50,000 μS/cm, if the conductivity of the sample fluid or the application suspension is below 5,000 μS/cm;
applying a flow of the application suspension to the measuring channel of the microfluidic particle analysis device; and
applying an alternating current from the current source to create an electric field in the operating space and monitoring an electrical signal between the first and the second electrode to detect target microparticle labelled with the electrically conducting nanoparticles from a phase of the electrical signal, and optionally also an amplitude of the electrical signal.
